SEQUENCE LISTING (4 sequences)

SEQ ID NO: 1 - protein, 15 aa, synthetic construct
HAEGTTSDVSSYLEG

SEQ ID NO: 2 - protein, 15 aa, synthetic construct
HAEGTFTSDSSYLEG

SEQ ID NO: 3 - protein, 16 aa, synthetic construct
HAEGTFTSDVSSYLEG

SEQ ID NO: 4 - protein, 22 aa, synthetic construct
DRLSSGKRINSASDDAAGLAIA

(19) United States
(12) Patent Application Publication
(10) Pub. No.: US 2025/0197441 A1
(43) Pub. Date: Jun. 19, 2025

(54) Safety-Catch Linkers for Solid Phase Peptide Synthesis

(71) Applicant: AAPPTec, LLC, Louisville, KY (US)

(72) Inventors: Fernando Albericio Palomera, Durban (ZA); Beatriz Garcia de la Torre, Durban (ZA); Sikabwe Noki, Durban (ZA); Hossain Saneii, Prospect, KY (US)

(73) Assignee: AAPPTec, LLC, Louisville, KY (US)

(21) Appl. No.: 18/545,556

(22) Filed: Dec. 19, 2023

(51) Int. Cl. C07K 1/04; C07C 319/20; C07C 323/62

(52) U.S. Cl. CPC C07K 1/042; C07C 319/20; C07C 323/62

(57) ABSTRACT

A safety-catch linker for solid phase peptide synthesis (SPPS) is provided, having a chemical structure according to Formula I:

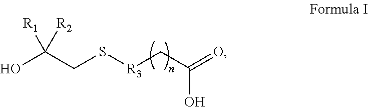

wherein R₁ and R₂ are each independently selected from hydrogen and methyl; R₃ is absent or phenyl; and n is 0 or 1. Also provided are methods of synthesizing the linker, methods of solid phase peptide synthesis (SPPS) of a target peptide employing the resin-bound linker, and kits for SPPS.

### SEQUENCE LISTING

A sequence listing having file name Sequence_Listing_AAX0001.xml (5 kB), created on Oct. 18, 2023, is incorporated herein by reference.

### TECHNICAL FIELD

The present disclosure relates to the field of solid phase peptide synthesis. Specifically, this disclosure relates to novel safety-catch linkers for coupling to a solid support and their methods of manufacture and use in solid phase peptide synthesis.

### BACKGROUND

Solid phase peptide synthesis (SPPS) is a method for chemical peptide synthesis wherein the nascent peptide is anchored via a linker at its carboxy terminus to an insoluble solid polymer support. Elongation of the peptide chain is carried out by iterative coupling of protected amino acids. Each amino acid addition, or cycle, includes steps of cleavage of the N-terminal protecting group, washing, coupling of an additional protected amino acid, and further washing to remove excess reagents. Because the growing peptide chain is anchored to the insoluble polymeric support, the excess reagents and soluble byproducts are removable by simple washing and filtration steps. SPPS has become the process of choice for synthesis of peptides for research and industrial purposes.

SPPS permits high volume production of linear peptides having 30 or more residues using two types of protecting groups labile to base (e.g., fluorenylmethoxycarbonyl, or Fmoc) and to acid (e.g., tert-butyl based protecting groups, such as tert-butyl, or tBu, tert-butyloxycarbonyl, or Boc, trityl, or Trt, and 2,2,4,6,7-pentamethyldihydrobenzofuran-5-sulfonyl, or Pbf). More complex peptides can also be manufactured via SPPS, through use of modified supports, reagents, and protecting groups. For example, efforts have been made to remove protecting groups with different catalysts, mild reducing agents, photolysis, and other means. However, none of these deprotection approaches is as straightforward as treatment with weak acids or bases.

Traditional SPPS methods employ fluorinated reagents such as trifluoroacetic acid (TFA), trifluoromethanesulfonic acid (TFMSA), trifluoroethanol (TFE), and hexafluoroisopropanol (HFIP) for the cleavage of unprotected (high content TFA, TFMSA) and protected (low content TFA, TFE, HFIP) peptides. However, these reagents are known to be potentially hazardous to the environment. For example, TFA resists biodegradation in water and has been shown to persist in the environment for years after exposure. Further, final peptide synthesis products often contain trace amounts of these fluorinated reagents, which may pose a health risk to humans or other organisms.

A need exists for additional SPPS reagents and strategies that minimize the presence of trace fluorinated reagents in peptide synthesis products and their environmental impact, while also permitting efficient cleavage and production of high-quality peptide products.

### SUMMARY

Accordingly, provided herein are novel thioethanol safety-catch linkers and their methods of manufacture and use in SPPS. The thioethanol linkers disclosed herein are stable to acid and base treatment until activated via oxidation, at which point the linkers become labile and release the target peptide product via a β-elimination reaction.

In one embodiment, a safety-catch linker for solid phase peptide synthesis (SPPS) is provided, having a chemical structure according to Formula I:

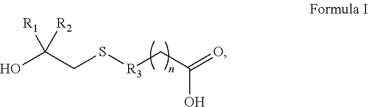

wherein: R₁ and R₂ are each independently selected from hydrogen and methyl; R₃ is absent or phenyl; and n is 0 or 1.

In another embodiment, a safety-catch linker for SPPS is provided, having a chemical structure according to Formula II:

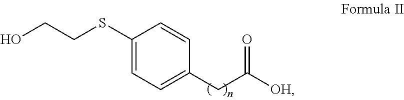

wherein n is 0 or 1.

In another embodiment, a method for synthesizing a safety-catch linker for solid phase peptide synthesis (SPPS) is provided, the method comprising: (a) providing a compound according to Formula III:

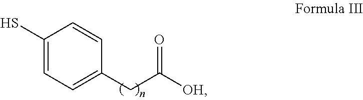

wherein n is 0 or 1; (b) reacting the compound of Formula III with MeOH and an acid catalyst to obtain a compound according to Formula IV:

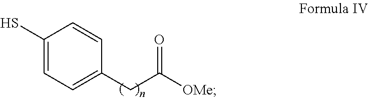

(c) reacting the compound of Formula IV with bromoethanol or choloroethanol in N, N-dimethylformamide (DMF) in the presence of Cs₂CO₃ to obtain the compound according to Formula V:

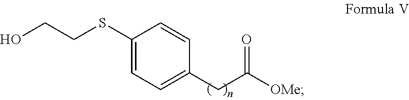

and (d) hydrolyzing the compound of Formula V to obtain the safety-catch linker according to Formula II:

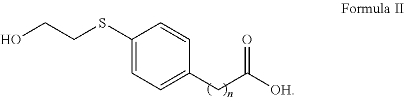

In another embodiment, a method for solid phase peptide synthesis (SPPS) of a target peptide is provided, the method comprising: (a) providing a solid support coupled to the safety-catch linker according to claim 1; (b) coupling a first amino acid having an N-terminal protecting group to the safety-catch linker via the C-terminus of the first amino acid to form an ester bond; (c) cleaving the N-terminal protecting group from the first amino acid; (d) coupling an additional amino acid to the N-terminus of the first amino acid; (e) repeating the coupling of step (d) as many times as necessary to provide an elongated peptide coupled to the solid support via the safety-catch linker; (f) oxidizing the product of step (e) to convert a sulfide moiety of the safety-catch linker to a sulfone moiety; (g) washing the product of step (f); and (h) cleaving the elongated peptide from the safety-catch linker and solid support via a beta elimination reaction to provide the target peptide.

In another embodiment, a kit for solid phase peptide synthesis (SPPS) is provided, comprising: (a) a solid support coupled to the safety-catch linker of claim 1; (b) a solution comprising an oxidizing agent; and (c) a solution comprising a secondary amine.

These and other features, aspects, and advantages will become better understood with reference to the following description and the appended claims.

Additional features and advantages of the embodiments described herein will be set forth in the detailed description that follows, and in part will be readily apparent to those skilled in the art from that description or recognized by practicing the embodiments described herein, including the detailed description that follows, the claims, as well as the appended drawings.

### BRIEF DESCRIPTION OF THE DRAWINGS

FIG. 1 is a high-performance liquid chromatography (HPLC) chromatogram for 4-((2-hydroxyethyl) thio) benzoic acid (Noki Linker 1).

FIG. 2 is a high resolution mass spectrometry (HRMS) spectrogram for 4-((2-hydroxyethyl) thio) benzoic acid (Noki Linker 1).

FIG. 3 is a proton nuclear magnetic resonance (¹HNMR) spectrum for 4-((2-hydroxyethyl) thio) benzoic acid (Noki Linker 1).

FIG. 4 is a carbon-13 nuclear magnetic resonance (¹³CNMR) spectrum for 4-((2-hydroxyethyl) thio) benzoic acid (Noki Linker 1).

FIG. 5 depicts synthetic Scheme 2, describing exemplary Fmoc and Boc SPPS protocols.

FIG. 6 depicts overlaid HPLC chromatograms for oxidation of Noki Linker with m-CPBA over time and under different reaction conditions, wherein line (a) depicts m-CPBA (1 eq.) at 10 min, line (b) depicts m-CPBA (2 eq.) at 10 min, line (c) depicts m-CPBA (2 eq.) at 30 min, and line (d) depicts m-CPBA (3 eq.) at 10 min.

FIG. 7A is an HPLC chromatogram for peptide Ac—Y(tBu)GGFL-OH (SEQ ID NO: 11).

FIG. 7B is an HPLC chromatogram for the depicted vinylsulfone obtained after cleavage of the target peptide.

FIG. 8A is an HPLC chromatogram for Leu-enkephalin peptide containing His, prepared with Noki resin.

FIG. 8B is an HPLC chromatogram for Leu-enkephalin peptide containing Trp, prepared with Noki resin.

FIG. 8C is an HPLC chromatogram for Leu-enkephalin peptide containing Cys, prepared with Noki resin.

FIG. 9 depicts an exemplary synthetic Scheme 5 for assessing formation of diketopiperazines (DKP) using Noki resin in conjunction with Boc and Fmoc protection under different reaction conditions (Protocol 1 and Protocol II) for test peptide Phe-D-Val-Pro.

FIG. 10 depicts overlaid HPLC chromatograms for the products of Scheme 5, wherein line (i) corresponds to Z-Phe-OH, line (ii) corresponds to Z-Phe-DVal-Pro-OH obtained using Boc and the in situ neutralization-coupling Protocol I, and line (iii) corresponds to product obtained using Fmoc-L-Phe-OH according to Protocol II.

FIG. 11A is an HPLC chromatogram of palmitoyl tripeptide Pal-KVK-OH synthesized using Noki resin.

FIG. 11B is an HPLC chromatogram of palmitoyl pentapeptide Pal-KTTKS-OH (SEQ ID NO: 15) synthesized using Noki resin.

FIG. 11C is an HPLC chromatogram of peptide-22 synthesized using Noki resin.

FIG. 12A is an HPLC chromatogram of peptide-22 synthesized with Noki resin using microwave synthesizer (CEM).

FIG. 12B is an HPLC chromatogram of peptide-22 synthesized with Wang resin using microwave synthesizer (CEM).

FIG. 13 is an HPLC chromatogram of H-HAEGTFTSDVSSYLEG-OH (SEQ ID NO: 3).

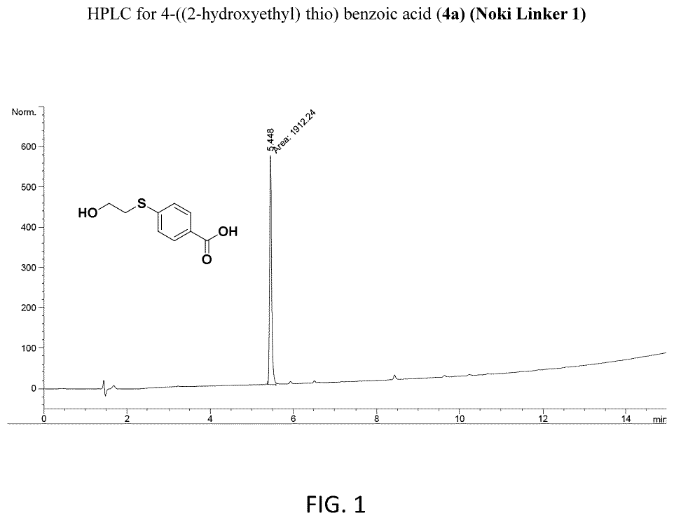

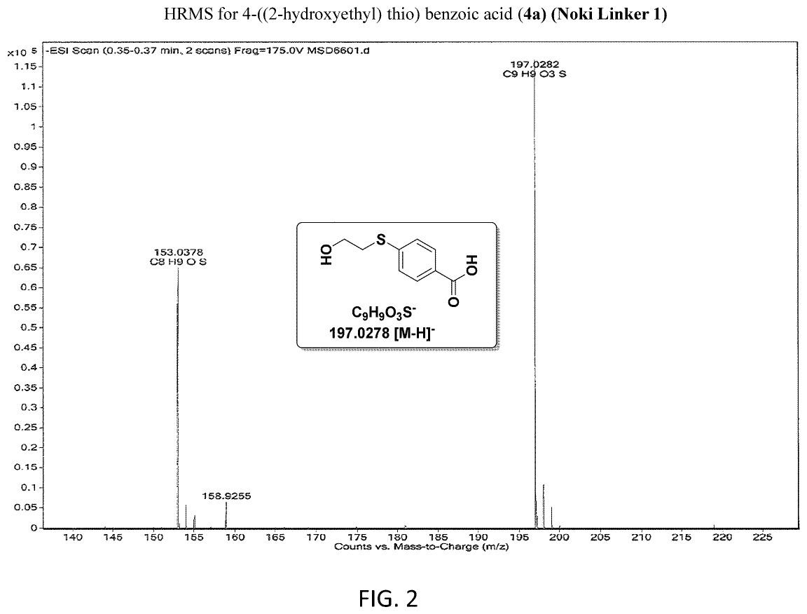

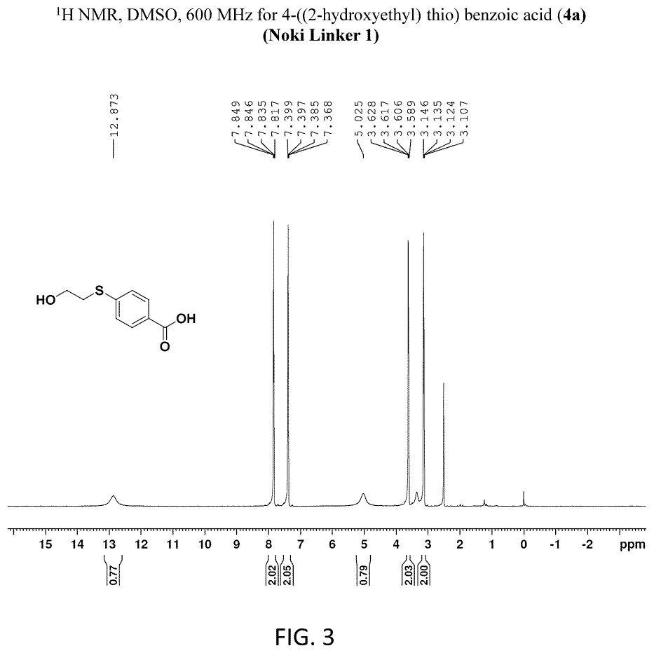

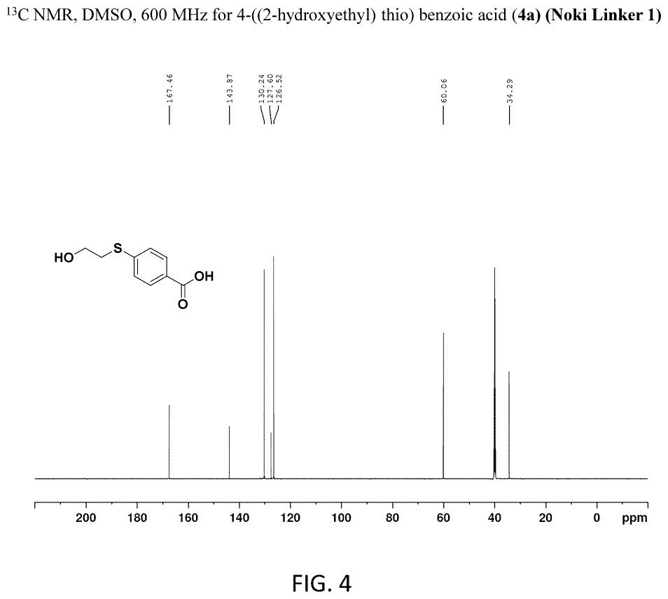

### DETAILED DESCRIPTION

The details of embodiments of the presently-disclosed subject matter are set forth in this document. Modifications to embodiments described in this document, and other embodiments, will be evident to those of ordinary skill in the art after a study of the information provided in this document.

While the following terms are believed to be well understood in the art, definitions are set forth to facilitate explanation of the presently-disclosed subject matter. Unless defined otherwise, all technical and scientific terms used herein have the same meaning as commonly understood by one of ordinary skill in the art to which the presently-disclosed subject matter belongs.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As used in this specification and the appended claims, the singular forms “a,” “an” and “the” include plural references unless the content clearly dictates otherwise.

### Safety-Catch Noki Linkers and Methods of Preparation

The term “safety-catch linker,” as used herein, refers to a linker for coupling a nascent peptide chain to a solid support during SPPS, wherein the linker is stable in the presence of acid and base treatment until it is activated, at which point the linker becomes labile in the presence of either acid or base. In specific embodiments disclosed herein, the safety-catch linker is a 2-hydroxyethylthio acid derivative that, upon oxidation, becomes labile to secondary amines through a beta elimination (β-elimination) reaction.

In embodiments, provided herein is a safety-catch linker for use in SPPS, having a chemical structure according to Formula I:

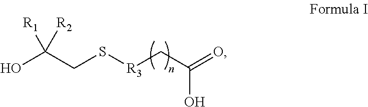

wherein:
R₁ and R₂ are each independently selected from hydrogen and methyl;
R₃ is absent or phenyl; and
n is 0 or 1.

In a more specific embodiment, provided herein is a safety-catch linker for use in SPPS, having a chemical structure according to Formula II:

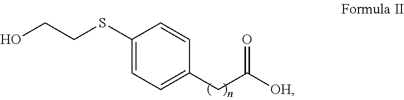

wherein n is 0 or 1.

In embodiments, the safety-catch linkers of Formula II are referred to as Noki Linker 1 and Noki Linker 2, having the following respective chemical structures:

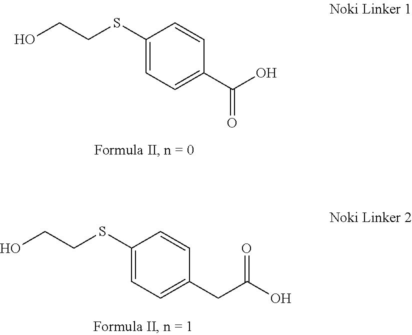 [SMILES: O=C(O)c1ccc(SCCO)cc1 ; O=C(O)Cc1ccc(SCCO)cc1]

While reference may be made to Noki Linker 1 or Noki Linker 2 throughout this disclosure, it should be appreciated that each of Noki Linker 1 and Noki Linker 2, as well as the compounds according to Formula I, are suitable for use in the methods and kits disclosed herein. Synthetic methods for preparing Noki Linker 1 and Noki Liker 2 are set forth in detail in Example 2 of this disclosure.

In embodiments, a solid support coupled to a safety-catch linker according to Formula I or Formula II is provided.

In embodiments, a method for synthesizing a safety-catch linker for SPPS is provided, the method comprising a first step (a) providing a compound according to Formula III:

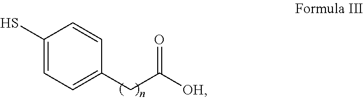

wherein n is 0 or 1, e.g., 4-mercaptobenzoic acid or 4-mercaptophenylacetic acid.

In a second step (b), the compound of Formula III is reacted with MeOH and an acid catalyst to obtain the methoxy compound according to Formula IV:

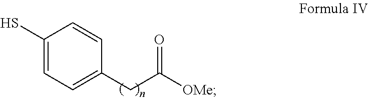

wherein n is 0 or 1.

Suitable acid catalysts for use in the reaction of step (b) include, but are not limited to, H₂SO₄, HCl, or other like acid catalysts known in the field.

In a third step (c), the compound of Formula IV is reacted with a halohydrin (e.g., bromoethanol, choloroethanol) in N, N-dimethylformamide (DMF) in the presence of Cs₂CO₃ to obtain the 2-hydroxyethylthio compound according to Formula V:

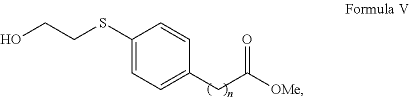

wherein n is 0 or 1.

In a fourth step (d), the compound of Formula V is hydrolyzed to obtain the safety-catch Noki linker according to Formula II:

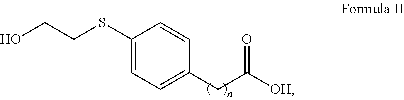

wherein n is 0 or 1.

Various hydrolyzation methods and reagents are suitable for use in step (d) of the synthetic method, as will be readily appreciated by the ordinary skilled person. In a specific embodiment, the hydrolyzing of step (d) is carried out with a base such as LiGH, NaOH, or KOH in tetrahydrofuran-water (THF-H₂O) in a ratio of 1:1.

### Methods for Solid Phase Peptide Synthesis of Target Peptides

The safety-catch Noki linkers disclosed herein are suitable for use in SPPS methods for the chemical synthesis of peptides, including peptides comprising sensitive amino acids (e.g., His, Trp).

In one embodiment, a method for SPPS of a target peptide is provided, the method comprising: (a) providing a solid support coupled to the safety-catch linker according to Formula I or Formula II; (b) coupling a first amino acid having an N-terminal protecting group to the safety-catch linker via the C-terminus of the first amino acid to form an ester bond; (c) cleaving the N-terminal protecting group from the first amino acid; (d) coupling an additional amino acid to the N-terminus of the first amino acid; (e) repeating the coupling of step (d) as many times as necessary to provide an elongated peptide coupled to the solid support via the safety-catch linker; (f) oxidizing the product of step (e) to convert a sulfide moiety of the safety-catch linker to a sulfone moiety; (g) washing the product of step (f); and (h) cleaving the elongated peptide from the safety-catch linker and solid support via a beta elimination reaction to provide the target peptide.

In general, any solid support may be used with the peptide synthesis methods and linkers disclosed herein. Suitable resins may be based on polystyrene, polystyrene-polyethylene glycol (PEG) composites, PEG, acrylamide PEG (PEGA), cross-linked ethoxylate acrylate (CLEAR), polyamides, polydimethylacrylamide, core pore glass (CPG), or any other support with the desired physical and chemical properties. In specific embodiments, the solid support is selected from the group consisting of amino methyl polysterene resin, polystyrene-PEG, PEG, polyamide, and CPG.

In a specific embodiment, the solid support is coupled to the safety-catch Noki linker by reacting the linker and the resin with a carbodiimide in the presence of a coupling additive. In a more specific embodiment, the linker and the resin are reacted with N,N′-diisopropylcarbodiimide (DIC) in the presence of 1-hydroxybenzotriazole (HOBt), dissolved in dimethylformamide (DMF). This coupling mixture serves to prevent double acylation of the safety-catch linker. Other coupling reagents and methods are known in the art and suitable for use in attaching the safety-catch linker to the solid support. For example, other suitable reagents include, but are not limited to, other carbodiimides, such as tert-butylethylcarbodiimide (TBEC), EDC, etc.; coupling additives such as Oxyma Pure, 1-Hydroxy-7-azabenzotriazole (HOAt), N-Hydroxysuccinimide (HOSu); uronium salts such as Hexafluorophosphate benzotriazole tetramethyl uronium (HBTU), Hexafluorophosphate azabenzotriazole tetramethyl uronium (HATU), 1-[(1-(Cyano-2-ethoxy-2-oxoethylideneaminooxy) dimethylaminomorpholino)]uronium hexafluorophosphate (COMU), 0-[(Ethoxycarbonyl)cyanomethylenamino]-N,N,N′,N′-tetramethyluronium hexafluorophosphate (HOTU), etc.; and phosphonium salts such as (Benzotriazol-1-yloxy)tripyrrolidinophosphonium hexafluorophosphate (PyBOP), (7-Azabenzotriazol-1-yloxy)tripyrrolidinophosphonium hexafluorophosphate (PyAOP), [Ethyl cyano(hydroxyimino)acetato-02]tri-1-pyrrolidinylphosphonium hexafluorophosphate (PyOxim), and the like.

In embodiments, washing steps are included at various steps of the method in order to remove excess reagents prior to the addition of an amino acid or the next step in the synthetic process. For example, in embodiments, washing steps are included after preparation of the Noki linker-resin support. Suitable wash reagents include, but are not limited to, DMF and DCM, optionally used in succession. In a very specific embodiment, the Noki linker-resin is washed in DMF and DCM prior to coupling a first amino acid to the support. Other washing reagents are known in the art and suitable for use. For example, other suitable washing reagents include N-butylpyrroldinome (NBP), dimethylsulfoxide (DMSO), ethyl acetate (EtOAc), 2-methyl-tetrahydrofurane, other green solvents, and combinations thereof.

In embodiments, coupling the first amino acid of step (b) comprises reacting the first amino acid and the product of step (a) with a carbodiimide (e.g., DIC) in the presence of N, N-dimethylaminopyridine (DMAP) or N-methylimidazole (NMI), dissolved in DMF. Other reagents are known in the art and suitable for use in coupling the first amino acid, including for example other carbodiimides or catalysts including but not limited to N-methylimidazole.

In embodiments, a washing step is carried out after coupling the first protected amino acid to the Noki linker-resin, in order to remove excess reagents. Suitable wash reagents include, but are not limited to, DCM and DMF, optionally used in succession. In a very specific embodiment, the product of step (b) is washed in DCM and DMF prior to cleaving the protective group from the first amino acid. Other washing reagents are known in the art and suitable for use. For example, other suitable reagents include N-butylpyrroldinome (NBP), dimethylsulfoxide (DMSO), ethyl acetate (EtOAc), 2-methyl-tetrahydrofurane, other green solvents, and combinations thereof.

In embodiments, the remaining hydroxy groups of the safety-catch linker are then capped by adding an acetylation reagent in the presence of a tertiary amine in DCM. In a specific embodiment, the hydroxy groups of the safety-catch linker are capped by adding Ac₂O and N,N-diisopropylethylamine (DIEA) in DCM, followed by a washing step in DCM and DMF, prior to deprotecting the N-terminus of the first amino acid.

In embodiments, the cleaving step (c) removes the N-terminal protecting group from the first protected amino acid. Various reagents and methods are suitable for deprotecting amino acids, and may be employed in the present methods depending on the particular protecting group to be removed. In a specific embodiment, the N-terminal protecting group is Fmoc and is removable by treating the construct with piperidine in DMF. Other suitable secondary amines include, but are not limited to, 4-methylpiperidine, piperazine, pyrrolidine, diazabicycloundecene (DBU), and the like.

Protecting groups suitable for use in protecting the N-terminus of the amino acids of the disclosed methods include, but are not limited to, Fmoc, tert-butyloxycarbonyl (Boc), N-allyloxycarbonyl (Alloc), p-nitrobenzyloxycarbonyl (pNZ), trityl (Trt), N-ε-1-(4,4-dimethyl-2,6-dioxocyclohex-1-ylidene)-3-methylbuty (ivDde), and the like. In a specific embodiment, the protecting group is an Fmoc or Boc moiety.

In embodiments, a washing step is carried out after deprotecting the first amino acid. Suitable wash reagents include, but are not limited to, DCM and DMF, optionally used in succession. In a very specific embodiment, the product of step (c) is washed in DCM and DMF prior to elongation of the peptide chain. Other suitable secondary amines include, but are not limited to, 4-methylpiperidine, piperazine, pyrrolidine, diazabicycloundecene (DBU), and the like.

Elongation of the peptide chain is carried out by step (d) coupling an additional amino acid to the N-terminus of the first amino acid; and step (e) repeating the coupling of step (d) as many times as necessary to provide the elongated peptide coupled to the solid support via the safety-catch linker. Various acylation mixtures are known in the art and suitable for use in elongation of the peptide chain as disclosed herein.

In embodiments, coupling the additional amino acid(s) of step (d) comprises reacting the N-protected additional amino acid to be added to the peptide chain and the product of step (c) with DIC-Oxyma Pure (ethyl(hydroxyimino)cyanoacetate) in the presence of dimethylformamide (DMF). However, the skilled artisan will appreciate that other methods of acylation are well known in the art and suitable for use in the present methods.

In a very specific embodiment, N-protected amino acid and ethyl(hydroxyimino)cyanoacetate (e.g., Oxyma Pure) are dissolved in DMF, DIC is added, and the mixture is added to the product of step (c) or step (d) to effect the addition of subsequent protected amino acids.

In embodiments, the additional amino acids are N-protected amino acids comprising a protecting group. In such cases, a deprotection step is required to deprotect the N-terminal amino acid prior to adding each subsequent protected amino acid to the growing peptide chain.

In a specific embodiment, an Fmoc protecting group is employed for N-terminal protection of first and subsequent amino acids, with the exception of the last amino acid, which is protected with a Boc protecting group.

Optionally, the amino acids of the peptides synthesized according to the disclosed methods may contain sidechains that are further protected by a protecting group. In such embodiments, the synthetic method may further comprise a step of deprotecting the amino acid side chains prior to activating the safety-catch Noki linker via oxidation, i.e., in step (f). Such deprotection may be carried out according to methods known in the art. For example, in embodiments a protecting group such as tBu may be removed by treatment with HCl in dioxane or other mineral acids.

In embodiments, the oxidation step (f) activates the safety-catch Noki linker by converting the sulfide moiety of the linker to a sulfone moiety, rendering the linker labile to secondary amines. In a specific embodiment, the oxidation step (f) comprises reacting the product of step (e) with meta-chloroperoxybenzoic acid (m-CPBA) in dichloromethane (CH₂Cl₂, DCM), although the skilled artisan will appreciate that other oxidation reagents are available and suitable for use in the present methods.

In embodiments, a washing step is carried out after oxidation of the Noki linker. Suitable wash reagents include, but are not limited to, DCM and DMF, optionally used in succession. In a very specific embodiment, the product of step (f) is washed in DCM and DMF prior to cleaving the peptide from the solid support.

In embodiments, the cleaving step (h) releases the elongated peptide from the safety-catch linker and solid support via a β-elimination reaction to provide the free target peptide. In an exemplary reaction, the target peptide is cleaved by addition of DEA in DCM, followed by a washing step to remove undesirable byproducts. The skilled artisan will appreciate that other β-elimination reagents are available and suitable for use in the present methods. In embodiments, a washing step with cold diethyl is repeated 3 or more times, after which the peptide product is dried. After drying, the peptide product may be dissolved in water, filtered, and lyophilized to provide the target peptide.

### Kits

In another embodiment, a kit for solid phase peptide synthesis (SPPS) is provided, the kit comprising: (a) a solid support coupled to the safety-catch linker Formula I or Formula II; (b) a solution comprising an oxidizing agent; and (c) a solution comprising a secondary amine. Additional reagents may also be included in embodiments of the kits disclosed herein.

In a specific embodiment, the oxidizing agent is selected from the group consisting of m-CPBA, H₂O₂, and the like.

In a specific embodiment, the secondary amine is selected from the group consisting of diethanolamine (DEA), piperidine, morpholine, 4-methylpiperidine, piperazine, and pyrrolidine. In a very specific embodiment, the secondary amine is DEA.

### EXAMPLES

The following examples are given by way of illustration are not intended to limit the scope of the disclosure.

### Example 1. Materials and Methods

All reagents and solvents were purchased from commercial suppliers and were used without further purification, unless otherwise stated. NMR spectra (¹H NMR and ¹³C NMR) were recorded on a Bruker AVANCE III 600 MHz spectrometer. Chemical shift values are expressed in parts per million (ppm). Analytical HPLC was performed on Agilent 1100 system using Phenomenex C18 column (3 m, 4.6×50 mm), and Chemstation software was used for data processing over a 5-95% gradient of CH₃CN (0.1% TFA)/H₂O (0.1% TFA) over 15 min, flow rate: 1.0 mL/min, detection at 220 nm. All mass spectrometry data were obtained from a Thermo Fisher Scientific UltiMate 3000 UHPLC-ISQ™ EC single quadrupole mass spectrometer in positive ion mode over a 5-95% gradient of MeCN (0.1% HCOOH)/H₂O (0.1% HCOOH) for 15 min unless otherwise specified. High resolution mass spectrometry (HRMS) was performed using an Agilent MSD-TOF mass spectrometer in positive-ion mode.

### Example 2. Noki Linker Synthesis

Two versions of the safety-catch linker (also referred to as Noki Linkers 1 and 2) were prepared starting from two commercially available mercaptophenyl acid derivatives: 4-mercaptobenzoic acid (1a) and 4-mercaptophenylacetic acid (1b), as shown in Schemes 1a and 1b:

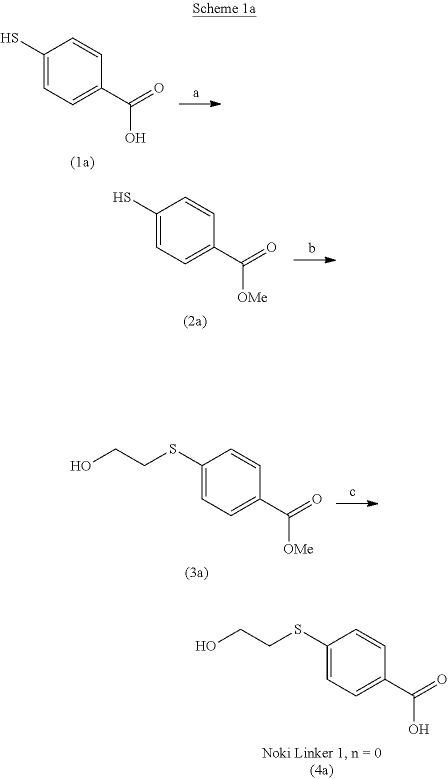 [SMILES: O=C(O)c1ccc(S)cc1 ; COC(=O)c1ccc(S)cc1 ; COC(=O)c1ccc(SCCO)cc1 ; O=C(O)c1ccc(SCCO)cc1]

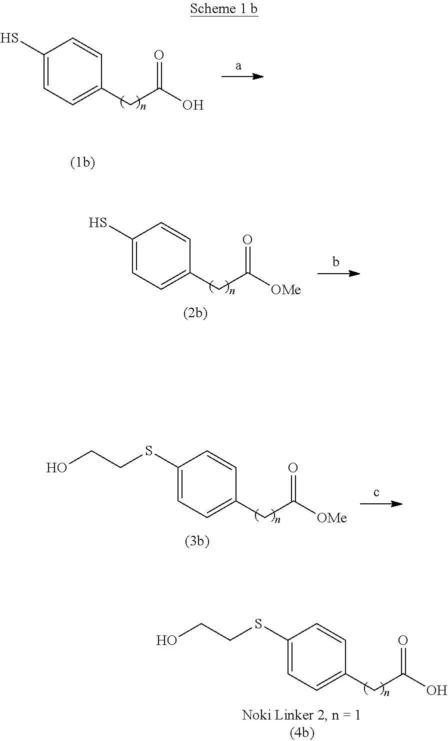

Reagents: (a) MeOH, H₂SO₄, 70° C., 2 hr; (b) bromoethanol, Cs₂CO₃, DMF, RT, overnight; (c) LiGH, THF-H₂O (1:1), room temperature (RT), overnight.

Briefly, the carboxylic group was protected as a methyl ester using methanol (MeOH) at 70° C. with an acid catalyst (H₂SO₄) for 2 h (2a, 2b). Both mercapto derivatives were then reacted with bromoethanol in N,N-dimethylformamide (DMF) in the presence of Cs₂CO₃ to render the corresponding methyl hydroxyethyl thio derivatives (3a, 3b). Finally, the methyl ester was hydrolyzed with LiOH in tetrahydrofuran-water (THF-H2O) (1:1) to provide the safety-catch linkers (4-((2-hydroxyethyl) thio) benzoic acid (4a) (Noki Linker 1) and 4-((2-hydroxyethyl) thio) phenylacetic acid (4b) (Noki Linker 2), with an overall yield of 90%. Preparation of Noki Linker 1 is described in greater herein detail below.

### (A) Preparation of methyl 4-mercaptobenzoate (2a)

A mixture of 4-mercaptobenzoic acid (1a) (5.0 g, 1 eq), H₂SO₄ (0.063 mL, 0.05 eq) in methanol (20 mL, 3.17 eq) was heated to 70° C. with stirring for 2 hours, the reaction mixture was monitoring by TLC (SiO₂) (MeOH/EtOAc, 5:95). The reaction mixture was cooled to 0° C. in ice bath, and then neutralized with NaHCO₃ to pH=7. Solvent was concentrated to about 10 mL. Then DCM (30 mL) and water (20 mL) was added. The aqueous phase was extracted with DCM (20 mL×3). The combined organics were dried over MgSO₄, filtered, and concentrated to render methyl 4-mercaptobenzoate (2a) (4.8 g) (96% yield).

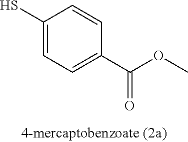

HPLC [5-95% of MeCN (0.1% TFA/H₂O (0.1% TFA) over 15 min] tR=8.493 min; ¹H NMR (600 MHz, DMSO): δ=7.81 (d, J=7.9 Hz, 2H; ArH), 7.43 (d, J=7.8 Hz, 6.01), (s, 2H, SH), 3.83 (s, 3H, CH₃), ¹³C{¹H}NMR (150 MHz, DMSO): 166.3, 140.8, 130.5, 130.1, 128.3, 126.7, 126.3, 52.4.

### (B) Preparation of methyl 4-((2-hydroxyethyl) thio) benzoate (3a)

A mixture of methyl 4-mercaptobenzoate (2a) (4.8 g), 2-bromoethanol (2.5 g) and Cs₂CO₃ (3.10 g) in N, N-dimethylformamide (DMF) (70 mL) was stirred at room temperature overnight. The mixture was filtered, and DCM (70 mL) was added. The solution was washed with water (50 mL×5), brine (50 mL×2), dried over MgSO₄, filtered, and concentrated. The crude product was purified by flash chromatography (silica gel; PE:EA=20:1 to 2:1) to render methyl 4-((2-hydroxyethyl) thio) benzoate (3a) (4,4 g) as a white solid (91% yield).

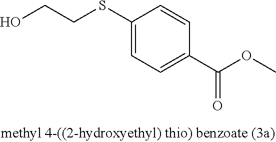

HPLC [5-95% of MeCN (0.1% TFA/H₂O (0.1% TFA) over 15 min] tR=7.078 min; ¹H NMR (600 MHz, DMSO): δ=7.85 (dd, J=6.3 Hz, 2H; ArH), 7. (dd, J=6.3 Hz, 2H, ArH), 5.04 (s, 1H, OH), 3.83 (s, 3H, CH₃), 3.62 (t, J=5.1 Hz, 2H), 3.14 (m, J=6.4 Hz, 2H)¹³C{¹H}NMR (150 MHz, DMSO): 166.4, 144.6, 130.1, 126.5, 126.3, 60.1, 52.4, 34.2. HRMS: m/z: calcd. for C₁₀H₁₃O₃S⁺: 213.0580 [M+H]⁺; found: 213.0577.

### (C) Preparation of 4-((2-hydroxyethyl) thio) benzoic acid (4a)

Lithium hydroxide monohydrate (1.366 g) in water (70 mL) was added into a solution of methyl 4-((2-hydroxyethyl) thio) benzoate (3a) (4.4 g) in tetrahydrofuran (THF) (70.0 mL). The reaction mixture was stirred at room temperature overnight. Solvent was removed under reduced pressure and the mixture was cooled in an ice bath and acidified to pH=1-2 with concentrated HCl. The aqueous phase was extracted with EtOAc (50 mL×5). The combined organic phases were washed with brine (50 mL×2), dried over MgSO₄, filtered, and concentrated to render 4-((2-hydroxyethyl) thio) benzoic acid (4a) (4.0 g) as a white solid (90% yield).

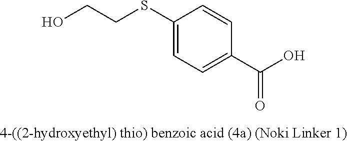 [SMILES: O=C(O)c1ccc(SCCO)cc1]

HPLC [5-95% of MeCN (0.1% TFA/H₂O (0.1% TFA) over 15 min] tR=5.448 min; ¹H NMR (600 MHz, DMSO): δ=12.8 (s, 1H, COOH), 7.84 (dd, J=8.3 Hz, 2H; ArH), 7.39 (dd, J=8.4 Hz, 2H, ArH), 5.03 (s, 1H, OH), 3.61 (dd, J=7.86 Hz, 2H, CH₂), 3.13 (dd, J=7.9 Hz, 2H, CH₂)¹³C{¹H}NMR (150 MHz, DMSO): 167.5, 143.9, 130.2, 127.6, 126.5, 60.1, 34.2. HRMS: m/z: calcd. for C₉H₉O₃S⁻: 197.0278 [M−H]⁻; found: 197.0282.

A like process was carried out for the synthesis of Noki Linker 2, using 4-mercaptophenylacetic acid as the starting reagent.

### Example 3. Exemplary Peptide Synthesis Protocol

### (A) Noki Linker Attachment to Amino Methyl Polysterene Resin

Amino methyl polysterene resin (0.1 mmol) was swollen in 5% DIEA-CH₂Cl₂ for 5 min, then the solvent was drained washed with CH₂Cl₂ (3×) and DMF (3×). Linker (2 eq.), HOBt (2 eq.) was dissolved in DMF, the mixture was added to the resin, followed by DIC (2 eq.). The mixture was put on shaker and agitated for 1 hr. The mixture was drained, and the resin beads washed with DMF (3×) and CH₂Cl₂ (3×) to provide Noki Linker-Resin.

### (B) Coupling First Amino Acid to the Noki Linker

Noki Linker-Resin (0.1 mmol) was swollen in DCM for 5 min, then the solvent was drained washed with CH₂Cl₂ (3×) and DMF (3×). Fmoc-AA-OH (5 eq.), DMAP (0.5 eq.) was dissolved in DMF, the mixture was added to the resin and DIC was added (5 eq.). The mixture was put on shaker and agitated for 2 hr. The mixture was drained, and the resin beads washed with DMF (3×) and CH₂Cl₂ (3×). The remaining hydroxy groups of the Noki Linker were capped adding Ac₂O (5 eq.), and DIEA in CH₂Cl₂ and the resin was agitated for 1 hr. The mixture was drained, and the resin beads washed with CH₂Cl₂ (3×) and DMF (3×).

### (C) Fmoc Cleavage

The N-terminal Fmoc protecting group was removed with a 20% solution of piperidine in DMF (2×5 min). The mixture was drained, and the resin beads washed with DMF (3×) and CH₂Cl₂ (3×).

### (D) Elongation of the Peptide

Fmoc-AA-OH (3 eq) and Oxyma Pure (3 eq) were dissolved in DMF, DIC (3 eq.) was added. After a pre-activation period of 3 min, the mixture was added to the resin, put on shaker, and agitated for 1 hr. The mixture was drained, and the resin beads washed with DMF (3×) and CH₂Cl₂ (3×).

(E) Cleavage from Noki Linker-Resin

The resin-bound peptide (prepared from 108 mg of about 0.1 mmol/g Noki Linker-Resin) was treated with 3 eq of meta-chloroperoxybenzoic acid (3 eq of m-CPBA), in DCM for 10 min at RT. The resin beads were washed with CH₂Cl₂ (3×) and DMF (3×). The resin-bound peptide was cleaved from resin using 80% DEA in CH₂Cl₂ put to the shaker and agitated for 30 min. Then cold diethyl ether was added to the cleavage mixture to remove unwanted side product. The washing was repeated three times, then dried. The product was dissolved in water and filtered, and the filtrate was lyophilized to obtained crude peptide.

### Example 4. Exemplary Peptide Synthesis Protocol Using Fmoc and Boc N-Terminal Protecting Groups

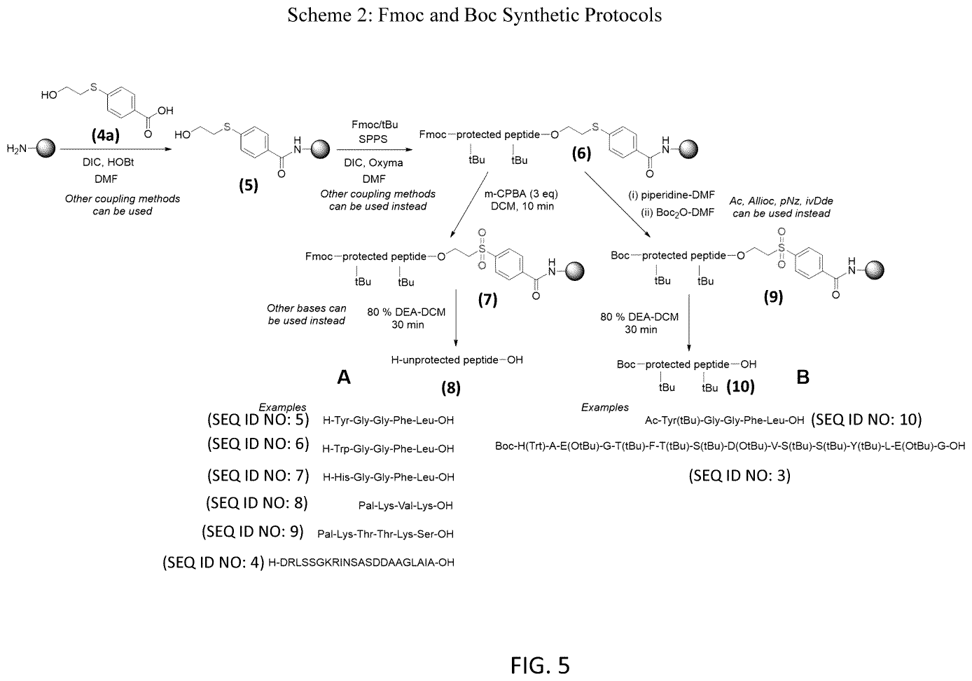

Scheme 2 (FIG. 5) depicts a synthetic scheme according to the present disclosure for SPPS using Fmoc (Path A) and BOC (Path B)N-terminal protecting groups. First, a Noki Linker is coupled to the solid support using HOBt dissolved in DMF, followed by DIC to provide Noki Linker-Resin. N-terminal Fmoc-protected amino acids, having side-chains protected with tBu, are added stepwise to the Noki Linker-Resin. N-terminal deprotection and coupling of successive amino acids may be carried out using DIC, Oxyma Pure, and DMF, although other coupling reagents are also suitable for use.

Referring to Path A of Scheme 2, the Noki Linker of the resin-bound, protected, elongated peptide may be activated by mCPBA in DCM to convert the sulfide to a sulfone. The peptide may then be cleaved from the Noki Linker-resin using DEA in CH₂Cl₂ (DCM). Exemplary peptides that may be synthesized according to Path A are set forth in Scheme 2.

Referring to Path B of Scheme 2, the N-terminal Fmoc-protected, Noki Linker-Resin-bound amino acid, having side-chains protected with tBu, is reacted with (i) piperidine and DMF and (ii) Boc₂O and DMF to provide an N-terminal Boc-protected, Noki Linker-Resin-bound amino acid, having side-chains protected with tBu, wherein the Noki Linker is activated to the sulfone form. This compound is then reacted with DEA in DCM to provide the cleaved, Boc-protected peptide having side-chains protected with tBu. Exemplary peptides that may be synthesized according to Path B are set forth in Scheme 2.

While Boc is exemplified in Path B of Scheme 2, it should be understood that other N-terminal protecting groups are also suitable for use in the method. Non-limiting examples of other suitable protecting groups include Ac, Alloc, pNz, ivDde, and the like.

### Example 5. Activation (Sulfide to Sulfone) of Noki Linker-Resin

The investigation into activation of the presently disclosed Noki Linkers was conducted in solid-phase peptide synthesis (SPPS) using a multi-detachable (double) linker strategy, which utilizes Rink amide resin with an attached linker in combination with a Noki Linker (0.1 mmol). The peptide linker (11) construct shown in Scheme 3 (below) was employed to examine the activation process, involving the oxidation of sulfide (thio) to sulfone, for the Noki Linker. Initially, a tripeptide, Gly-Phe-Leu (GFL), was synthesized on Rink amide resin utilizing Fmoc chemistry. Then, Noki Linker was introduced, and the full sequence of Leu-enkephalin (Tyr-Gly-Gly-Phe-Leu, SEQ ID NO: 5) was extended from the Noki Linker, with acetylation of the α-amino terminal of Tyr, using Fmoc/tBu chemistry once again (Y(tBu)GGFL, SEQ ID NO: 11).

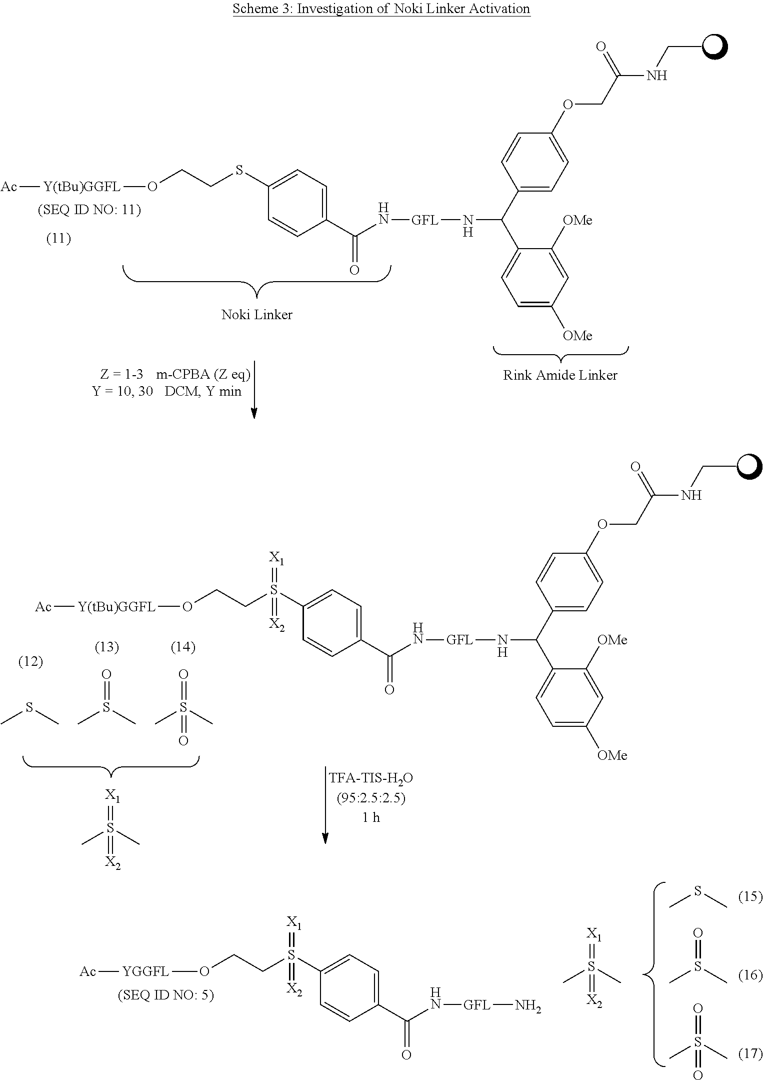

Sill referring to Scheme 3, using Ac-Tyr(tBu)-Gly-Gly-Phe-Leu-O-Noki-Linker-Gly-Phe-Leu-NH-Rink amide-resin (11) as a substrate, the oxidation of the sulfide to sulfone was studied using meta-chloroperoxybenzoic acid (m-CPBA) in dichloromethane (DCM). Various amounts of m-CPBA (1-3 eq.) and reaction times (10-30 min) were used. In cases where oxidation did not occur, the reduced form (12) was produced. The presence of the sulfone derivative (14) indicated complete oxidation, whereas the presence of the product (13) signified a partial oxidation leading to the corresponding sulfoxide.

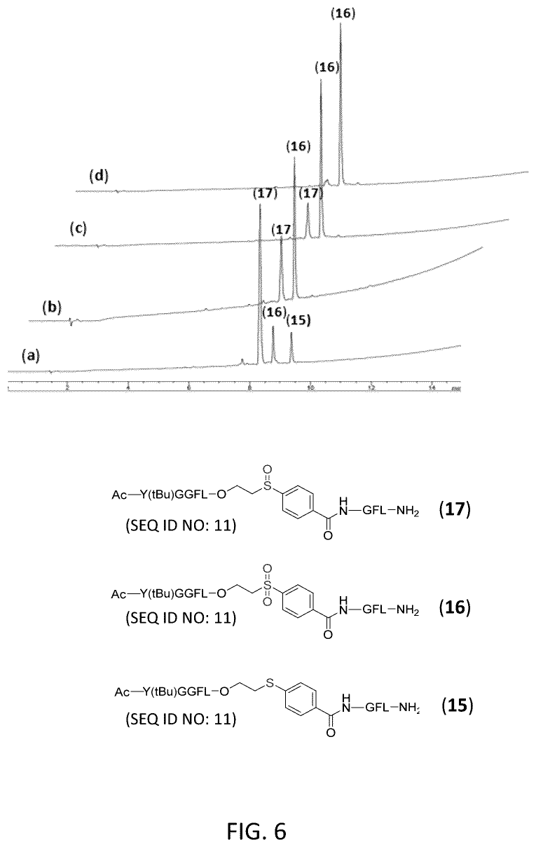

m-CPBA (1 eq.) in defect was employed to identify the corresponding sulfoxide. FIG. 6, line (a) displays a major peak indicating the incorporation of a single oxygen atom (sulfoxide) (17), accompanied by roughly equivalent amounts of the double oxidation product (sulfone) (16) and the initial starting material (sulfide) (15). This implies the complete consumption of all m-CPBA within 10 minutes. Subsequently, 2 eq. of m-CPBA were utilized, and after 10 minutes, the principal peak corresponded to the sulfone derivative (16), with the sulfoxide derivative (17) accounting for approximately half of the total, as shown in FIG. 6, line (b). By the 30-minute mark shown in FIG. 6, line (c), the peak corresponding to the targeted sulfone derivative (16) showed a slight increase, while the presence of the sulfoxide derivative (17) remained noticeable (approx. 25%). Finally, nearly quantitative conversion was achieved using 3 eq. (1.5 excess) of m-CPBA within 30 minutes (FIG. 6, line (d)). To conclude, the solid-phase oxidation of the sulfide to sulfone proceeds cleanly, without affecting the peptide chain, using moderate excesses of m-CPBA (1.5 excess), and within a relatively brief timeframe (10-30 minutes).

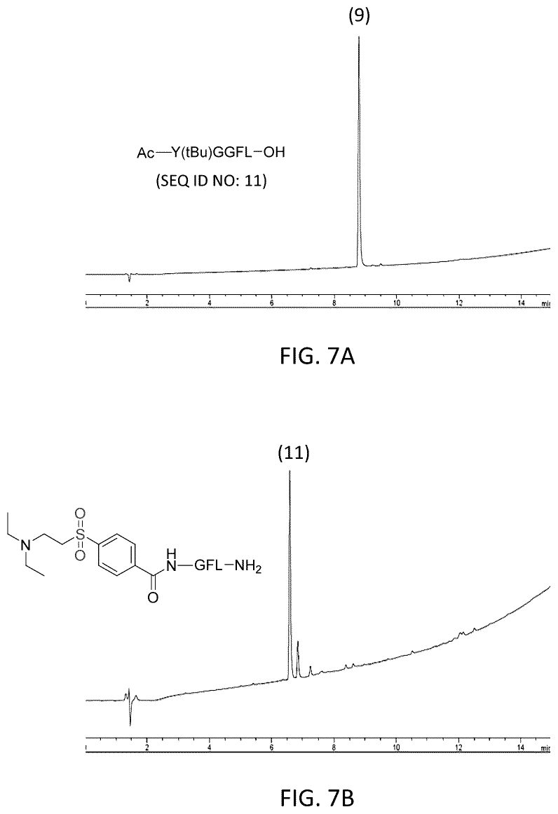

To confirm these results, the protected Ac-Leu-enkephalin peptide [Ac-Tyr(tBu)-Gly-Gy-Phe-Leu-OH (SEQ ID NO: 11)] was cleaved from the resin using 80% DEA in DCM. Then cold diethyl ether was added to the cleavage mixture to remove unwanted side product. The washing was repeated three times, then dried. The product was dissolved in water and filtered, and the filtrate was lyophilized to obtained crude peptide. The HPLC chromatogram (FIG. 7A) showed the target peptide with an excellent purity. Furthermore, the remaining peptide oxidized resin was treated with TFA in the presence of scavengers and the HPLC chromatogram showed cleanly the adduct of the DEA on the vinylsulfone obtained after the cleavage (FIG. 7B).

### Example 6. Peptide Cleavage from Activated Noki Linker

The oxidized Noki Linker-containing resin (also referred to as Noki resin) (14) was subjected to various DEA and secondary amine solutions in DCM, as shown in Scheme 4:

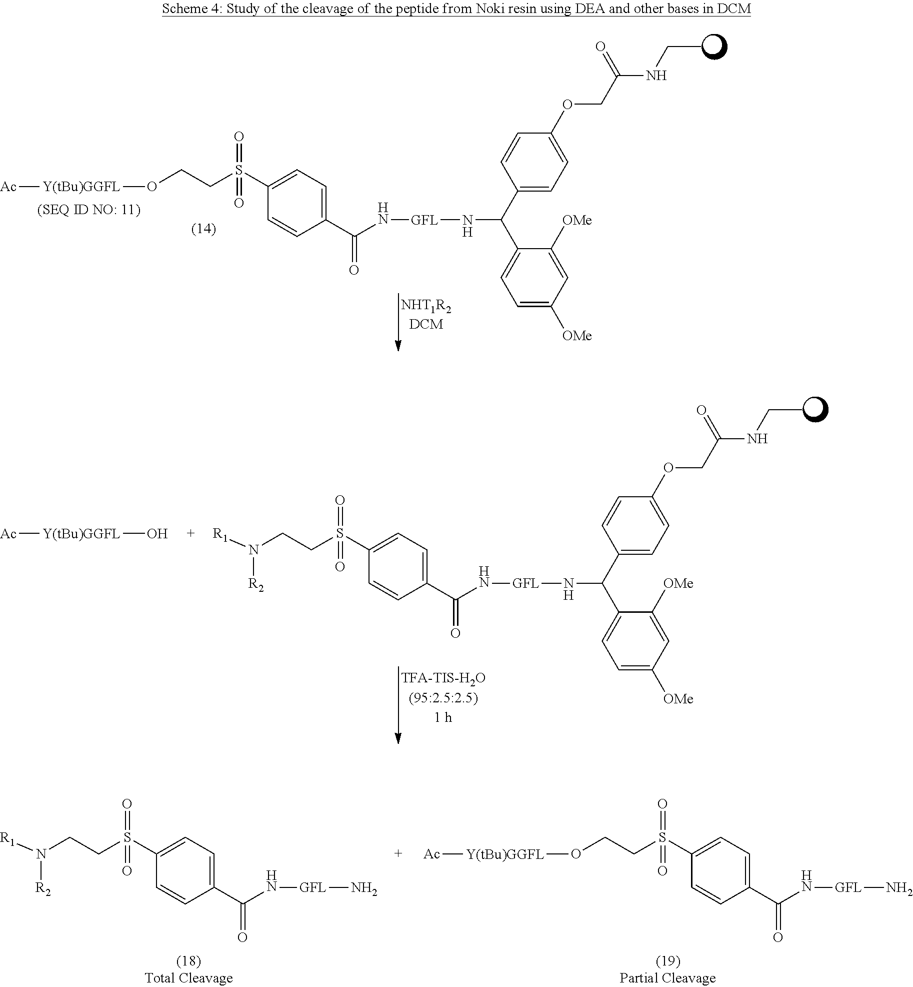

First, DEA was selected over piperidine, which is the preferred amine for Fmoc removal, as well as other secondary amines, due to its lower boiling point (55.6° C. for DEA), compared to piperidine (106° C.) and morpholine (129° C.). Furthermore, it is important to note that DEA is not subjected to the same regulatory restrictions as piperidine. The outcomes presented in Table 1 indicate that even a 50% concentration of DEA in DCM induces an excellent peptide detachment within 30 minutes.

TABLE 1
HPLC analysis of cleavage study, to determined
percentage recovery of peptide.

| | Protected Peptides |
|---|---|
| | (18 vs. 19) |
| | Cleavage of |
| Cleavage Basic conditions, | Ac-Y(tBu)GGFL-OH)/(%) |
| rt, 30 min | (SEQ ID NO: 11) |
| 100% DEA | 100 |
| 80% DEA-DCM | 100 |
| 50% DEA-DCM | 100 |
| 20% DEA-DCM | 63 |
| 5% DEA-DCM | 15 |
| 10% Piperidine-DMF | 96 |
| 10% 4-Methylpiperidine-DMF | 93 |
| 60% Morpholine-DMF | 47 |

### Example 7. Synthesis of Peptides with Sensitive Amino Acids (His, Trp) with m-CPBA Oxidizing Reagent

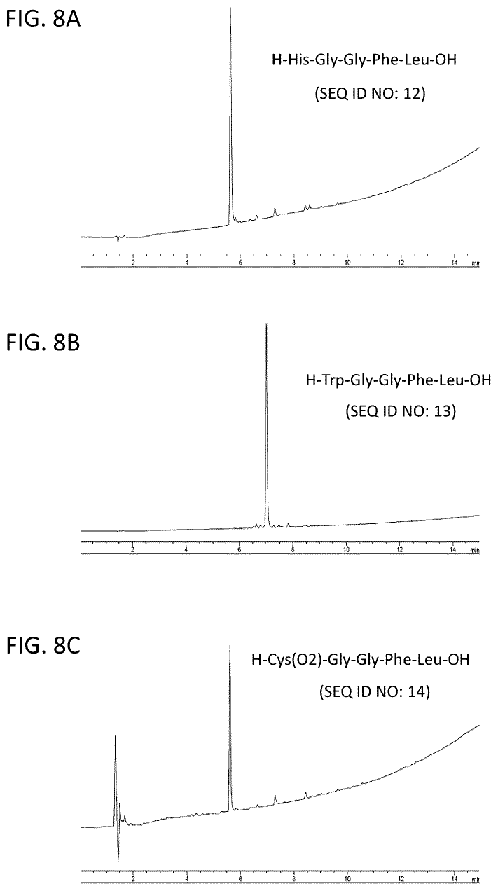

As will be appreciated, a Noki Linker strategy is not compatible with Met-containing peptides, because the oxidizing conditions will also render the corresponding sulfone of the Met, which cannot be reduced back to the sulfide. To test the compatibility of the presently disclosed linkers and methods of synthesis with other sensitive amino acid-containing peptides, protected Leu-enkephalin peptides containing His(Trt), Trp(Boc), and Cys(Trt) were synthesized following the standard Fmoc chemistry. For His and Trp containing peptides, 3 eq. m-CPBA in DCM for 10 min rendered peptides of more than acceptable purity (FIGS. 8A, 8B). On the other hand, the Cys peptide even protected with the hindered trityl (Trt) group is cleanly converted to the sulfonic acid derivative as shown by its m/z (528.22, which represents a mass increase of +32, from the expected m/z of 496.22) (FIG. 8C). Use of a Cys disulfide protecting group such as sec-isoamyl mercaptan (SIT) was also not compatible with the oxidation.

### Example 8. Compatibility of Noki-Resin with Boc Chemistry, Minimizing DKP Formation

In addition to being stable to bases, Noki Linker resin is also stable to treatments with acid such as TFA and is therefore compatible with Boc chemistry. Although Fmoc chemistry generally has more advantages than the Boc counterpart, in certain instances Boc protection may be desirable for protection of the α-amino function.

Sequences are prone to give diketopiperazines (DKPs) where the C-terminal amino acid is Pro or Gly and the subsequent amino acid has the D-configuration. Pro and (to a lesser extent) Gly favor the cis-configuration of the peptide bond and the six-member ring DKP containing one L and one D residue is more stable than when the two amino acids are of the same configuration. Both factors are determinants for the formation of the DKP. Although this reaction can take place in an acid medium, it is more severe in the presence of bases, such as piperidine treatment to remove the Fmoc group of the second amino acid, and even during the neutralization step after the removal of the Boc group of the same amino acid. The minimization of this side-reaction can take place when the second amino acid is incorporated with the α-amino protected with Boc and, after the removal of the Boc with TFA, the neutralization is avoided. Instead, an in situ neutralization-coupling strategy is performed. Thus, the third protected amino acid is activated and added to the trifluoroacetyl amino resin in the presence of a base such as DIEA. Once the salt is neutralized the acylation step with the third amino acid competes with the cyclization, thereby minimizing DKP formation. Although, this strategy cannot be applied with TFA-sensitive resins, such as Wang resins, variants of the strategy using Trt, Alloc, or pNz for the α-amino function of the second amino acid with Wang resins have been developed.

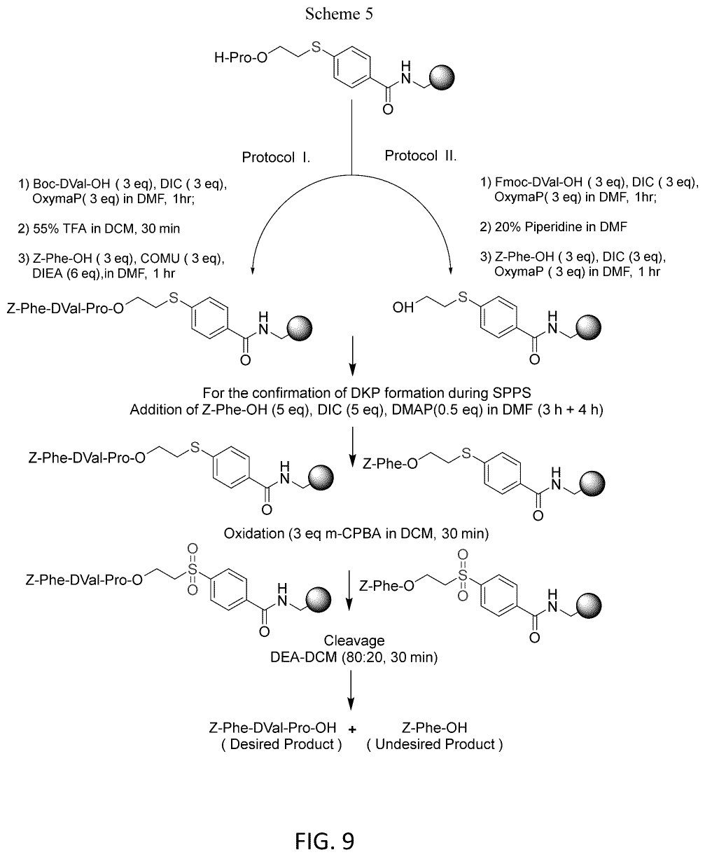

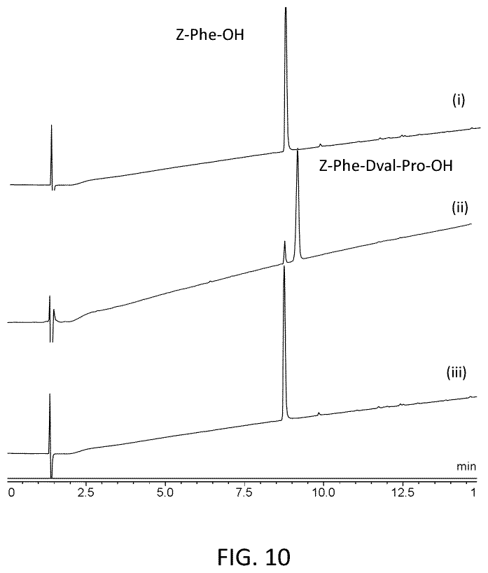

Using as a model the dipeptide D-Val-L-Pro, the formation of DKP using Noki resin in conjunction with Boc and Fmoc protection for the D-Val was studied. In the case of Boc, the incorporation of the third amino acid, Z-Phe-OH, was done following an in situ neutralization-coupling protocol, while the Z-Phe-OH was incorporated to the Fmoc-synthesis using a regular DIC and Oyxma Pure coupling protocol. If DKP has been formed, the DKP release has provoked the formation of the initial hydroxy-resin, which can be acylated in low extension with the Z-L-Phe-OH (the esterification is more demanding than the amide formation and requires DMAP as catalyst). To acylate completely the potential hydroxy groups formed, an extra acylation with Z-L-Phe-OH, DIC, and DMAP is carried out in both resins. Oxidation of the resin followed by DEA treatment should render the tripeptide Z-L-Phe-D-Val-L-Pro-OH when DKP is not formed, or just Z-L-Phe-OH in the case of DKP formation. FIG. 9 shows the scheme followed (Scheme 5) and the HPLC profiles of peptides obtained from both experiments are shown in FIG. 10. In the case of the synthesis carried out with Fmoc-D-Val-OH, only Z-Phe-OH is obtained (line (i)), which shows a 100% DKP formation. On the other hand, when Boc and in situ neutralization-coupling is performed, only 15% of the Z-Phe-OH is formed (line (ii)). These results agree with others obtained using similar in situ neutralization-coupling methods with other protecting groups and/or resins (data not shown).

Formation of DKPs was not detected during the synthesis of peptides not containing C-terminal dipeptides prone to give this side reaction.

### Example 9. Synthesis of Unprotected Peptides Free of Trifluoroacetyl Salts

The use of fluorinated reagents such as TFA, TFMSA, TFE, or HFIP for the cleavage of unprotected peptides (high content of TFA and TFMSA) or protected peptides (low content of TFA, TFE, HFIP) is associated with two main problems in the context of sustainability and environmental safety. First, the waste generated from these compounds is potentially hazardous and poses an environmental risk. Second, the use of these reagents favors a final target peptide that contains substantial or trace amounts of fluorinated compounds.

The use of Noki-resin minimizes the use of these reagents. When used, the reagents can be employed in earlier steps of the upstream process in order to minimize the presence of fluorinated contaminants in the final peptide product. To demonstrate this approach, palmitoyl tripeptide and pentapeptide (commonly used in cosmetics as anti-aging agents) and peptide-22 were selected for synthesis.

For these peptides, after elongation of the peptide, oxidation, and removal of the side-chain protecting groups with TFA, the peptide resin is gently washed with 5% DIEA in DCM before the final cleavage of the peptide with DEA. The washings with DIEA will remove the trifluoro acetyl salts before the cleavage of the peptide from the resin with DEA. Even with peptides containing Lys (Boc), Ser, Thr, Tyr, Asp, and Glu protected with tBu groups (e.g., palmitoyl tripeptide/pentapeptide-5), the protecting groups can be removed with HCl-dioxane, thereby avoiding the use of TFA entirely.

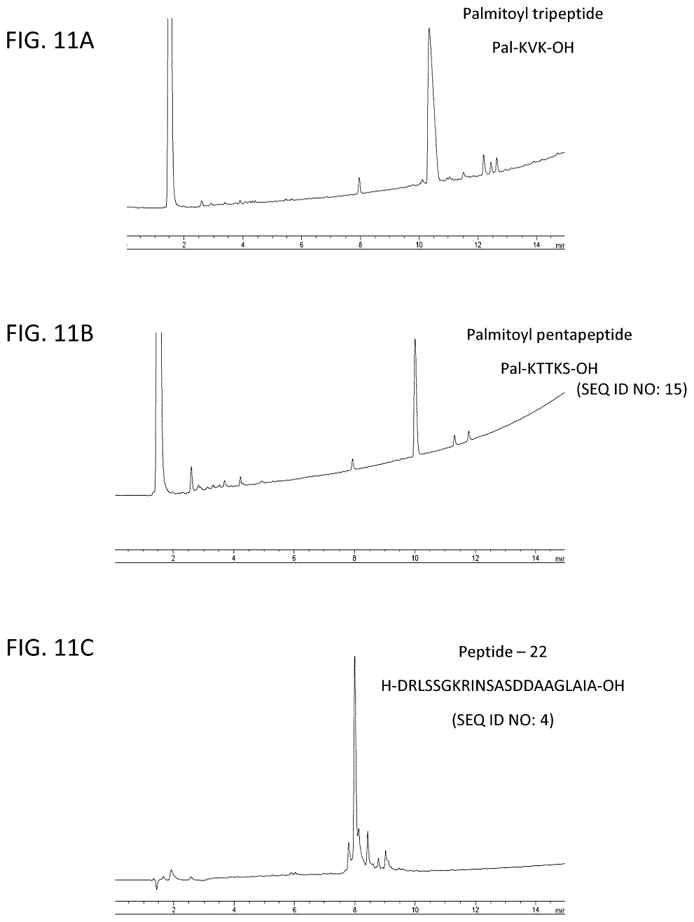

FIGS. 11A, 111B, and 11C show the HPLC profiles of palmitoyl tripeptide, pentapeptide-5, and peptide-22 using Noki resin via the following method: (i) Fmoc/tBu peptide elongation using DIC-Oxyma Pure (3 eq. of each one, 1 hr coupling); (ii) oxidation of the thio to the sulfone with m-CPBA (3 eq.) in DCM for 10 min; (iii) side-chain deprotection with TFA-triisopropylsilane (TIS)-H₂O (90:5:5) for 1 hr followed by DMF-DCM washing; (iv) neutralization with 5% DIEA in DCM; and (v) cleavage with 80% DEA in DCM for 30 min. Peptide-Noki-resin after incorporation of 3, 5, and 22 Fmoc-amino acids were cleaved from the resin and analyzed by HPLC.

Cleavage of peptide-22 was carried out with DEA. After the DEA treatment, cold ether was added to the cleavage cocktail containing the resin and the peptide was precipitated. The supernatant was removed by decantation after centrifugation, and cold ether was added again, repeating the process two more times. Finally, peptide-22 was extracted with H₂O and lyophilized. As the two small palmitoyl peptides did not precipitate when cold ether was added, the supernatant after cleavage was removed by evaporation with an N₂ stream, ether was added and removed with an N₂ stream (repeated twice), and the peptide was extracted with 10% aqueous acetic acid and lyophilized. The small peaks around 6 min and between 12 and 13 min do not correspond to peptide material (FIGS. 11A, 11B).

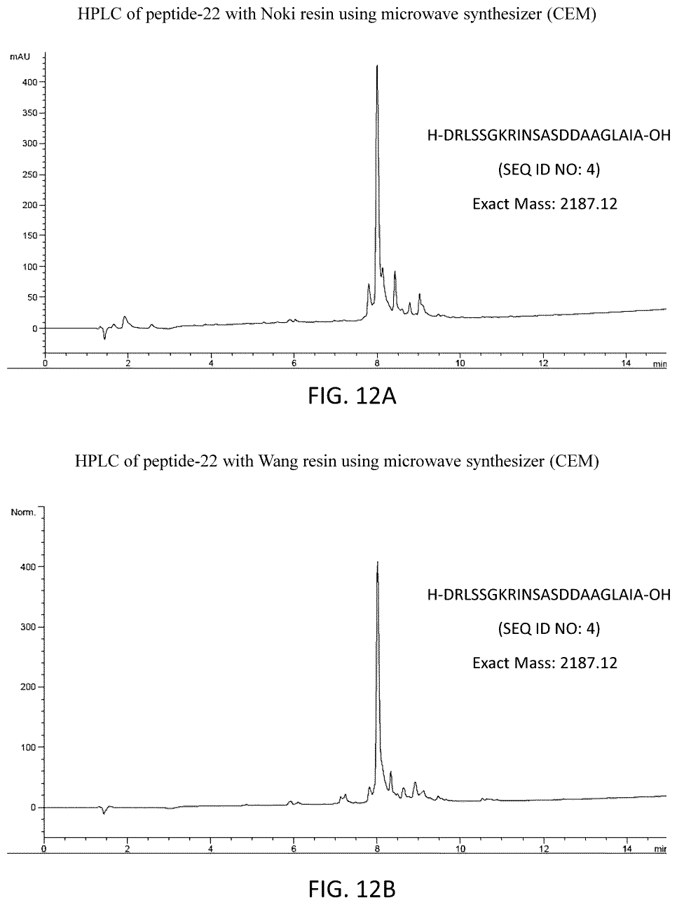

Synthesis of peptide-22 was repeated using a CEM microwave-assisted automatic synthesizer using a regular program. The synthesis of the same peptide was also carried out with the CEM synthesizer using a Wang-resin. In both cases, the first three amino acids were incorporated manually to minimize the risk of the DKP formation although the C-terminal dipeptide L-Ile-L-Ala does not show apriori risk of racemization. In the case of the Wang resin, the peptide was cleaved with TFA-TIS-H₂O (95:2.5:2.5) for 1 hr. Both syntheses yielded a similar purity profile (FIGS. 12A and 12B). The results indicate that the Noki resin is compatible with microwave synthesis.

### Example 10. Synthesis of Protected Peptides

In addition of the sequential synthesis of peptides, the preparation of long peptides such as the GLP-1 agonists, liraglutide, semaglutide, or tirzepatide, can be carried out in a convergent way using a hybrid solid-phase and solution strategy: synthesis of protected peptides having the free carboxylic group on solid phase, following by its assembling in solution. The synthesis of protected peptides is typically carried out using chloro-tritylchloride (CTC) resin, which allows the release of the protected peptide using low TFA content (1-2%) (for C-terminal amide peptides, the Sieber resin is used, cleaving the peptide with 4% of TFA). After cleavage of the protected peptides from the CTC-resin, the process should be carried out carefully, as an increase in the TFA concentration can provoke the undesired removal of the protecting groups. In this context, the development of a free acid method for the preparation of protected peptides is desirable.

Noki resin is a suitable candidate for the preparation of protected peptides because the Noki resin allows the release of the peptide without the use of TFA. In such a synthesis, the elongation of the peptide chain incorporates Fmoc-amino acids, except for the last amino acid, which is protected with Boc if it is the N-terminal that is protected, or in form of Alloc or pNz if the central portion is protected. After the elongation, oxidation of the linker and cleavage with DEA renders the protected peptide. As a model, the 16-amino acid protected peptide corresponding to the C-terminal sequence of liraglutide was synthesized.

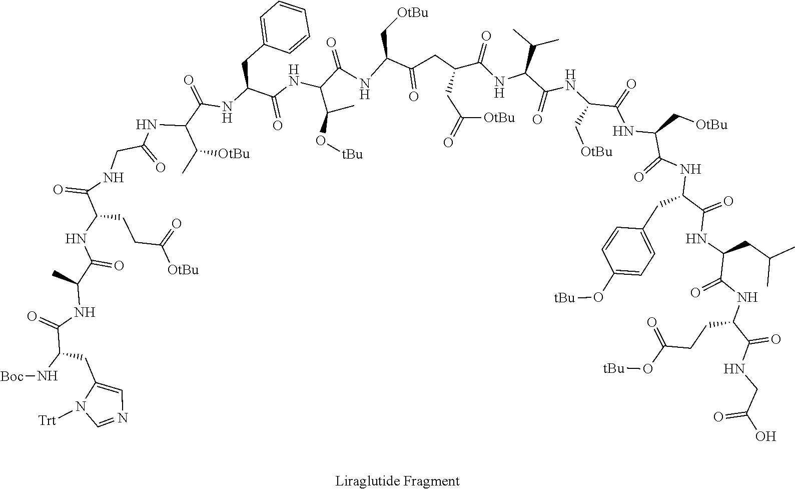

The 16-amino acid-protected peptide (HAEGTFTSDVSSYLEG, SEQ ID NO: 3) was elongated on the Noki resin and, after incorporation of the last Fmoc-His (Trt)-OH, the Fmoc was removed and the α-amino acid was re-protected in form of Boc with Boc₂O. Next, the protected peptide-Noki-resin was treated with 80% DEA and the solution was poured into cold H₂O, which resulted in precipitation of the protected peptide. DCM and DEA were removed in the rotavapor under reduced pressure and the protected peptide was collected after centrifugation and decantation or was directly lyophilized.

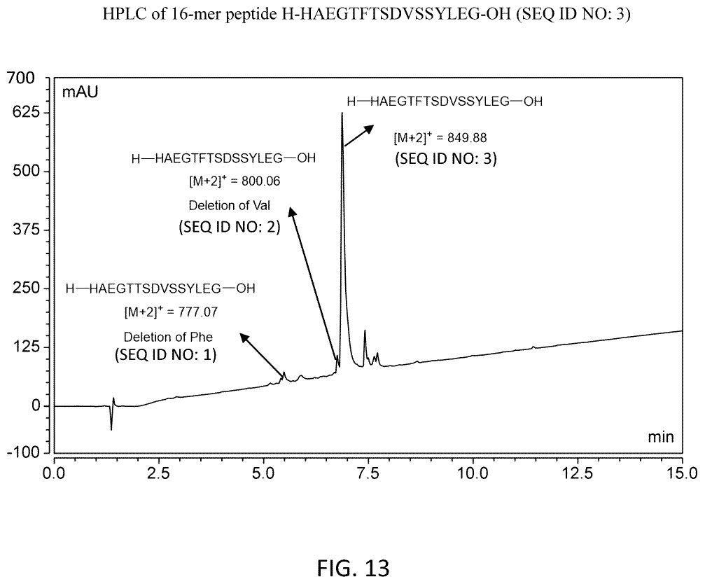

The protected peptide was treated in solution with TFA-TIS-H₂O (95:2.5:2.5) for 1 hr and the unprotected peptide was analyzed by HPLC. FIG. 13 shows HPLC results. The quality of the crude was acceptable, considering that previous synthesis of the same sequence required the concourse of three pseudo-Pro dipeptides. The main impurities were Phe (HAEGTTSDVSSYLEG, SEQ ID NO: 1) and Val (HAEGTFTSDSSYLEG, SEQ ID NO: 2) deletions, which could be avoided by optimizing synthesis parameters.

### Example 11. Exemplary Linkers

Exemplary linkers according to Formula I are set forth in the following table:

| Compound | Structure | Name |
|---|---|---|
| Noki Linker 1 | 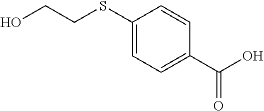 [SMILES: O=C(O)c1ccc(SCCO)cc1] | 4-((2-hydroxyethyl)thio) benzoic acid |
| Noki Linker 2 | 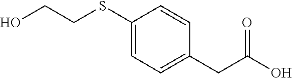 [SMILES: O=C(O)Cc1ccc(SCCO)cc1] | 4-((2-hydroxyethyl)thio) phenylacetic acid |
| 101 | 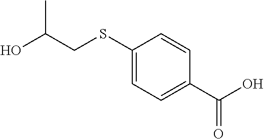 | 4-((2-hydroxypropyl)thio) benzoic acid |
| 102 | 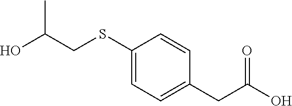 | 2-(4-((2-hydroxypropyl) thio)phenyl)acetic acid |
| 103 | 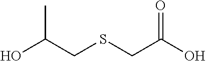 | 2-((2-hydroxypropyl)thio) acetic acid |
| 104 | 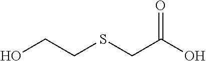 | 2-((2-hydroxyethyl)thio) acetic acid |
| 105 | 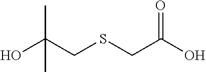 | 2-((2-hydroxy-2- methylpropyl)thio)acetic acid |
| 106 | 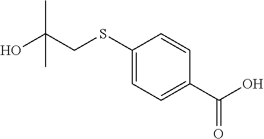 | 4-((2-hydroxy-2- methylpropyl)thio)benzoic acid |
| 107 | 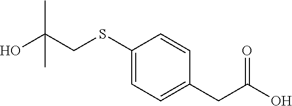 [SMILES: CC(C)(O)CSc1ccc(CC(=O)O)cc1] | 2-(4-((2-hydroxy-2- methylpropyl)thio)phenyl) acetic acid |

Aspects of the present disclosure can be described with reference to the following numbered clauses, with specific features laid out in dependent clauses.

1. A safety-catch linker for solid phase peptide synthesis (SPPS), having a chemical structure according to Formula I:

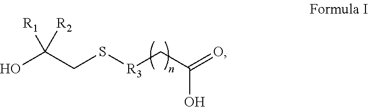

wherein: R₁ and R₂ are each independently selected from hydrogen and methyl; R₃ is absent or phenyl; and n is 0 or 1.
   2. The safety-catch linker for SPPS according to clause 1, having a chemical structure according to Formula II:

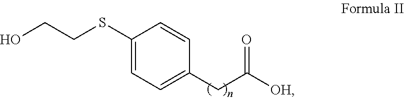

wherein n is 0 or 1.
3. A method for synthesizing a safety-catch linker for solid phase peptide synthesis (SPPS), the method comprising: (a) providing a compound according to Formula III:

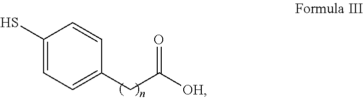

wherein n is 0 or 1; (b) reacting the compound of Formula III with MeOH or an acid catalyst to obtain a compound according to Formula IV:

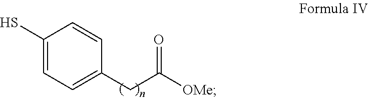

(c) reacting the compound of Formula IV with bromoethanol or choloroethanol in N, N-dimethylformamide (DMF) in the presence of Cs₂CO₃ to obtain the compound according to Formula V:

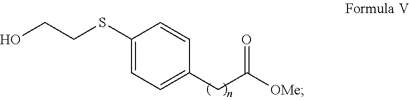

and
(d) hydrolyzing the compound of Formula V to obtain the safety-catch linker according to Formula II:

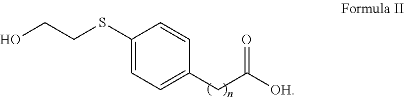

4. The method according to clause 3, wherein the acid catalyst of step (b) is selected from H₂SO₄ and HCl.
5. The method according to clause 3 or clause 4, wherein the hydrolyzing of step (d) is carried out with a reagent selected from LiGH, NaOH, or KOH, in tetrahydrofuran-water (THF-H₂O) in a ratio of 1:1.
6. A method for solid phase peptide synthesis (SPPS) of a target peptide, the method comprising: (a) providing a solid support coupled to the safety-catch linker according to clause 1; (b) coupling a first amino acid having an N-terminal protecting group to the safety-catch linker via the C-terminus of the first amino acid to form an ester bond; (c) cleaving the N-terminal protecting group from the first amino acid; (d) coupling an additional amino acid to the N-terminus of the first amino acid; (e) repeating the coupling of step (d) as many times as necessary to provide an elongated peptide coupled to the solid support via the safety-catch linker; (f) oxidizing the product of step (e) to convert a sulfide moiety of the safety-catch linker to a sulfone moiety; (g) washing the product of step (f); and (h) cleaving the elongated peptide from the safety-catch linker and solid support via a beta elimination reaction to provide the target peptide.
7. The method according to clause 6, wherein the solid support is selected from the group consisting of amino methyl polysterene resin, polystyrene-PEG, PEG, polyamide, and core pore glass (CPG).
8. The method according to clause 6 or clause 7, wherein the solid support is coupled to the safety-catch linker by reacting the safety-catch linker and the solid support with N,N′-diisopropylcarbodiimide (DIC) in the presence of 1-hydroxybenzotriazole (HOBt).
9. The method according to any of clauses 6-8, wherein coupling the first amino acid of step (b) comprises reacting the first amino acid and the product of step (a) with DIC in the presence of N, N-dimethylaminopyridine (DMAP).
10. The method according to any of clauses 6-9, wherein any of the first or additional amino acids has a protected sidechain.
11. The method according to clause 10, further comprising deprotecting the side chains prior to the oxidizing of step (f).
12. The method according to any of clauses 6-11, wherein coupling the additional amino acid of step (d) comprises reacting the additional amino acid and the product of step (c) with DIC-Oxyma Pure in the presence of dimethylformamide (DMF).
13. The method according to any of clauses 6-12, wherein the oxidizing of step (f) comprises reacting the product of step (e) with meta-chloroperoxybenzoic acid (m-CPBA) in dichloromethane (DCM).
14. The method according to any of clauses 6-13, wherein the washing of step (g) comprises washing the product of step (f) separately with DCM and DMF.
15. The method according to any of clauses 6-14, wherein the cleaving of step (h) comprises reacting the product of step (g) with a secondary amine.
16. The method according to clause 15, wherein the secondary amine is selected from the group consisting of diethanolamine (DEA), piperidine, morpholine, 4-methylpiperidine, piperazine, pyrrolidine, and diazabicycloundecene (DBU).
17. The method according to any of clauses 6-16, wherein trifluoroacetic acid (TFA) is not employed as a reagent in any step of the method.
18. The method according to any of clauses 6-17, further comprising the step of:
(i) washing the product of step (h) in diethyl ether to provide a target peptide that is substantially free of byproducts.
   19. The method according to any of clauses 6-18, wherein the N-terminal protecting group is selected from fluorenylmethylcarbonyl (Fmoc), tert-butyl (tBu), tert-butyloxycarbonyl (Boc), N-allyloxycarbonyl (Alloc), p-nitrobenzyloxycarbonyl (pNZ), trityl (Trt), and N-ε-1-(4,4-dimethyl-2,6-dioxocyclohex-1-ylidene)-3-methylbuty (ivDde).
   20. The method according to any of clauses 6-19, wherein the solid support is substantially insoluble.
   21. A kit for solid phase peptide synthesis (SPPS), comprising:
(a) a solid support coupled to the safety-catch linker of clause 1 or clause 2;
(b) a solution comprising an oxidizing agent; and
(c) a solution comprising a secondary amine.
   22. The kit according to clause 21, wherein the oxidizing agent is selected from the group consisting of m-CPBA and H₂O₂.
   23. The kit according to clause 21 or clause 22, wherein the secondary amine is selected from the group consisting of diethanolamine (DEA), piperidine, morpholine, 4-methylpiperidine, piperazine, pyrrolidine, and diazabicycloundecene (DBU).
   24. The kit according to any of clauses 21-23, wherein the safety-catch linker is

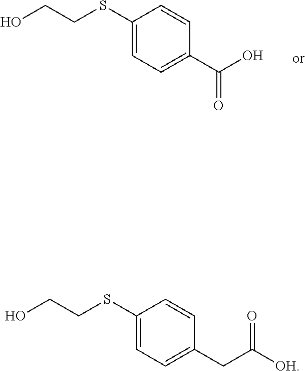 [SMILES: O=C(O)c1ccc(SCCO)cc1 ; O=C(O)Cc1ccc(SCCO)cc1]

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue. The term “substantially” is used herein also to represent the degree by which a quantitative representation may vary from a stated reference without resulting in a change in the basic function of the subject matter at issue. Thus, it is used to represent the inherent degree of uncertainty that may be attributed to any quantitative comparison, value, measurement, or other representation, referring to an arrangement of elements or features that, while in theory would be expected to exhibit exact correspondence or behavior, may in practice embody something less than exact.

It is noted that one or more of the following claims utilize the term “wherein” as a transitional phrase. For the purposes of defining the present technology, it is noted that this term is introduced in the claims as an open-ended transitional phrase that is used to introduce a recitation of a series of characteristics of the structure and should be interpreted in like manner as the more commonly used open-ended preamble term “comprising.”

It should be understood that where a first component is described as “comprising” or “including” a second component, it is contemplated that, in some embodiments, the first component “consists” or “consists essentially of” the second component. Additionally, the term “consisting essentially of” is used in this disclosure to refer to quantitative values that do not materially affect the basic and novel characteristic(s) of the disclosure.

It should be understood that any two quantitative values assigned to a property or measurement may constitute a range of that property or measurement, and all combinations of ranges formed from all stated quantitative values of a given property or measurement are contemplated in this disclosure.

While particular embodiments have been illustrated and described herein, it should be understood that various other changes and modifications may be made without departing from scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A safety-catch linker for solid phase peptide synthesis (SPPS), having a chemical structure according to Formula I: 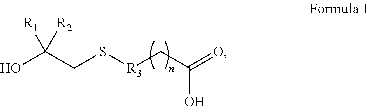
wherein:
R₁ and R₂ are each independently selected from hydrogen and methyl;
R₃ is absent or phenyl; and
n is 0 or 1.

2. The safety-catch linker for SPPS according to claim 1, having a chemical structure according to Formula II: 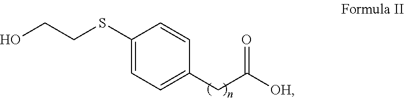
wherein n is 0 or 1.

3. A method for synthesizing a safety-catch linker for solid phase peptide synthesis (SPPS), the method comprising:
(a) providing a compound according to Formula III: 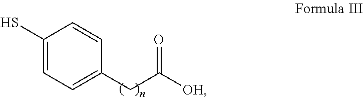
wherein n is 0 or 1;
(b) reacting the compound of Formula III with MeOH and an acid catalyst to obtain a compound according to Formula IV: 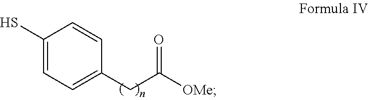
(c) reacting the compound of Formula IV with bromoethanol or choloroethanol in N, N-dimethylformamide (DMF) in the presence of Cs₂CO₃ to obtain the compound according to Formula V: 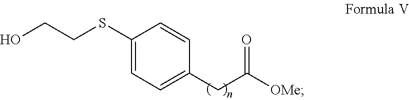
and
(d) hydrolyzing the compound of Formula V to obtain the safety-catch linker according to Formula II:

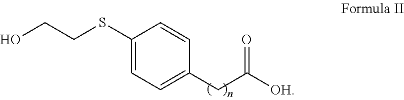

4. The method according to claim 3, wherein the acid catalyst of step (b) is selected from H₂SO₄ and HCl.

5. The method according to claim 3, wherein the hydrolyzing of step (d) is carried out with a reagent selected from LiGH, NaOH, or KOH, in tetrahydrofuran-water (THF-H₂O) in a ratio of 1:1.

6. A method for solid phase peptide synthesis (SPPS) of a target peptide, the method comprising:
(a) providing a solid support coupled to the safety-catch linker according to claim 1;
(b) coupling a first amino acid having an N-terminal protecting group to the safety-catch linker via the C-terminus of the first amino acid to form an ester bond;
(c) cleaving the N-terminal protecting group from the first amino acid;
(d) coupling an additional amino acid to the N-terminus of the first amino acid;
(e) repeating the coupling of step (d) as many times as necessary to provide an elongated peptide coupled to the solid support via the safety-catch linker;
(f) oxidizing the product of step (e) to convert a sulfide moiety of the safety-catch linker to a sulfone moiety;
(g) washing the product of step (f); and
(h) cleaving the elongated peptide from the safety-catch linker and solid support via a beta elimination reaction to provide the target peptide.

7. The method according to claim 6, wherein the solid support is selected from the group consisting of amino methyl polysterene resin, polystyrene-PEG, PEG, polyamide, and core pore glass (CPG).

8. The method according to claim 6, wherein the solid support is coupled to the safety-catch linker by reacting the safety-catch linker and the solid support with N,N′-diisopropylcarbodiimide (DIC) in the presence of 1-hydroxybenzotriazole (HOBt).

9. The method according to claim 6, wherein coupling the first amino acid of step (b) comprises reacting the first amino acid and the product of step (a) with DIC in the presence of N, N-dimethylaminopyridine (DMAP).

10. The method according to claim 6, wherein any of the first or additional amino acids has a protected sidechain.

11. The method according to claim 10, further comprising deprotecting the side chains prior to the oxidizing of step (f).

12. The method according to claim 6, wherein coupling the additional amino acid of step (d) comprises reacting the additional amino acid and the product of step (c) with DIC-Oxyma Pure in the presence of dimethylformamide (DMF).

13. The method according to claim 6, wherein the oxidizing of step (f) comprises reacting the product of step (e) with meta-chloroperoxybenzoic acid (m-CPBA) in dichloromethane (DCM).

14. The method according to claim 6, wherein the washing of step (g) comprises washing the product of step (f) separately with DCM and DMF.

15. The method according to claim 6, wherein the cleaving of step (h) comprises reacting the product of step (g) with a secondary amine.

16. The method according to claim 15, wherein the secondary amine is selected from the group consisting of diethanolamine (DEA), piperidine, morpholine, 4-methylpiperidine, piperazine, pyrrolidine, and diazabicycloundecene (DBU).

17. The method according to claim 6, wherein trifluoroacetic acid (TFA) is not employed as a reagent in any step of the method.

18. The method according to claim 6, further comprising the step of:
(i) washing the product of step (h) in diethyl ether to provide a target peptide that is substantially free of byproducts.

19. The method according to claim 6, wherein the N-terminal protecting group is selected from fluorenylmethylcarbonyl (Fmoc), tert-butyl (tBu), tert-butyloxycarbonyl (Boc), N-allyloxycarbonyl (Alloc), p-nitrobenzyloxycarbonyl (pNZ), trityl (Trt), and N-ε-1-(4,4-dimethyl-2,6-dioxocyclohex-1-ylidene)-3-methylbuty (ivDde).

20. The method according to claim 6, wherein the solid support is substantially insoluble.

21. A kit for solid phase peptide synthesis (SPPS), comprising:
(a) a solid support coupled to the safety-catch linker of claim 1;
(b) a solution comprising an oxidizing agent; and
(c) a solution comprising a secondary amine.

22. The kit according to claim 21, wherein the oxidizing agent is selected from the group consisting of m-CPBA and H₂O₂.

23. The kit according to claim 21, wherein the secondary amine is selected from the group consisting of diethanolamine (DEA), piperidine, morpholine, 4-methylpiperidine, piperazine, pyrrolidine, and diazabicycloundecene (DBU).

24. The kit according to claim 21, wherein the safety-catch linker is 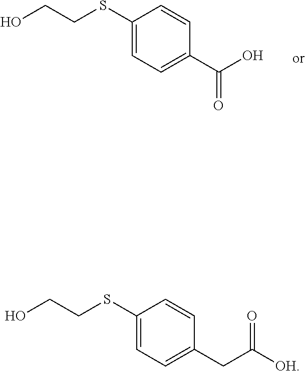 [SMILES: O=C(O)c1ccc(SCCO)cc1 ; O=C(O)Cc1ccc(SCCO)cc1]
